(19) United States
(12) Patent Application Publication
(10) Pub. No.: US 2024/0341610 A1
(43) Pub. Date: Oct. 17, 2024

(54) IMPLANT, LIVING BODY MONITORING SYSTEM, AND BIOMEDICAL MANAGEMENT SYSTEM

(71) Applicant: ATONARP INC., Minato-ku, Tokyo (JP)

(72) Inventor: Prakash Sreedhar MURTHY, Minato-ku, Tokyo (JP)

(73) Assignee: ATONARP INC., Minato-ku, Tokyo (JP)

(21) Appl. No.: 18/552,696

(22) PCT Filed: Mar. 18, 2022
(86) PCT No.: PCT/JP2022/012498

§ 371 Date: Feb. 13, 2024

(30) Foreign Application Priority Data
Apr. 13, 2021 (JP) 2021-067980

(51) Int. Cl. A61B 5/026; A61B 5/00; A61B 5/06

(52) U.S. Cl. CPC A61B 5/0261; A61B 5/062; A61B 5/4839; A61B 5/686; A61B 2562/0238

(57) ABSTRACT

There is provided a living body monitoring system equipped with: an implant that is embedded under the skin of the living body and includes an optical part with a reflective function that is embedded below a target under the skin; and an analyzer apparatus. The analyzer apparatus includes: an irradiating apparatus for irradiating a blood vessel that is a target under the skin of the organism with a laser; and a detector that detects scattered light (CARS light) from the blood vessel via the optical unit of the implanted implant. A biomedical management system equipped with a medication system may also be provided.

### TECHNICAL FIELD

The present invention relates to an implant to be embedded in a living body, such as a human, and to a system for monitoring a living body (biomonitoring system) and a system for managing a living body (biomedical management system) using the same.

### BACKGROUND ART

International Publication WO2014/178199 describes the provision of a monitor that monitors the internal state of a living body (an organism) from the surface of the organism. The monitor includes: a probe including an observation window which is attached to the surface of the organism; a unit that irradiates, with a laser, at least part of an observation area on the surface of the organism accessed through the observation window; a unit that detects scattered light caused by the laser irradiation from each of a plurality of observation spots that are two-dimensionally dispersed across the observation area; a Doppler analyzer unit and a SORS analyzer unit that limits, based on the scattered light obtained from the plurality of observation spots, to a first observation spot that has been determined to produce scattered light containing information on a target part inside the organism, out of the plurality of observation spots; and a CARS analysis unit that acquires a spectroscopic spectrum for at least one component from the first observation spot or observation spots in a periphery thereof and outputs first information indicating an internal condition of the organism based on intensities in the spectroscopic spectrum.

### SUMMARY OF INVENTION

When light, for example, laser light, is used from outside a living body, such as outside the skin, to access a target of measurement, treatment, or the like located inside the living body, focusing light on the target with a sufficiently high intensity may involve the risk that the intensity (illuminance or irradiation intensity) per area when the light passes through the skin might be too high and adversely affect the skin and the living body (human body). On the other hand, if the light could not be focused on the target with sufficiently high intensity, it might be difficult to perform the desired measurement or treatment.

As one example, when trying to accurately and non-invasively acquire information on the inside of a living body, typically various information included in blood flowing through a blood vessel, using scattered light obtained by irradiating a target with one or more laser lights, it is desirable to obtain scattered light of sufficient intensity from the target. Although it would be better to increase the intensity of the irradiating laser lights for this purpose, in consideration of the effect on the skin and/or the human body, there is also demand for the intensities of the irradiating laser lights to be suppressed.

One aspect of the present invention is an implant to be embedded (inserted, implanted) under the skin of a living body (an organism). This implant includes an optical part (optical unit) that guides light irradiated from outside the skin toward a target under the skin and/or guides light from the target toward the outside of the skin. With this implant, it is possible to control the direction in which light propagates under the skin (that is, inside the living body), to focus light toward the target inside the living body, and to guide light from the target out of the skin. The target may be tissue under the skin, such as a capillary under the epidermis, may be tissue, such as a blood vessel, present in the dermis or subdermal subcutaneous tissue, or may be a blood vessel or tissue at a deeper location.

The implant may have an optical part that includes a reflective function, for example, a reflective surface, and may be implanted (embedded, inserted) under the skin so as to be positioned below the target (that is, on the opposite side of the target to the skin). If the target is blood flowing through a blood vessel, the implant may be embedded so that the optical part guides light irradiated from outside the skin toward the blood vessel and/or guides light from the blood in the blood vessel toward the outside of the skin. This implant makes it possible to control the direction of light (scattered light) emitted from the target under the skin within the living body, and to improve the intensity of the scattered light collected by a non-invasive analyzer apparatus. Alternatively, to obtain scattered light, the implant may control the direction of light (one or more laser lights) irradiated from outside the skin inside the living body to focus the lights onto the target. The implant may guide the lasers so as to guide the scattered light produced at the target mainly in a direction out of the skin. This makes it possible to accurately acquire, via light, information on a target in the living body, for example, various information included in the blood flowing through a blood vessel.

This implant may be used in combination with an analyzer apparatus that includes an irradiating apparatus that irradiates a target under the skin of a living body with a laser and a detector that detects scattered light from the target via the optical part of the embedded implant. Even with an analyzer apparatus that is attached on the skin of the living body, the scattered light emitted in various directions from the target under the skin will be reflected toward the skin by the optical part of the implant inserted under the target in the living body (in vivo), which enables efficient detection outside the skin and improves the intensity of the scattered light used for analyzing the target. In addition, scattered light in the forward direction that has a high scattering intensity can be reflected by the implant and guided out of the skin, which improves the intensity of the scattered light used for analysis.

This implant may be used in combination with an analyzer apparatus including an irradiating apparatus that irradiates a target with a laser via the optical part of the implant embedded under the skin of a living body and a detector that detects scattered light from the target. With an analyzer apparatus that is attached to the skin of the living body, the intensity of the laser irradiated onto the skin can be suppressed by focusing the laser onto the target via an implant inside the living body (in vivo), which suppresses influences of the laser on the skin. In addition, the target under the skin can be irradiated with the laser from below via the implant disposed below the target, so that the forward direction where the intensity of the scattered light from the target is high can be set in a direction out of the skin. Accordingly, in the analyzer apparatus disposed outside the skin, it is possible to perform measurement using scattered light in the forward direction that has high scattering intensity.

The implant may further include at least one support portion that partially protrudes from the perimeter of the optical part to maintain the position inside the living body of the optical part. The implant may also include a marker (marker substance) that enables contactless detection of the position of the optical part inside the living body from outside the living body. One example of a marker is a magnetic material. A typical target for which an implant is installed is blood in a blood vessel, with an implant being favorable for detecting and analyzing the components of the blood flowing through the blood vessel. The target may also be other tissue, such as a lymphatic vessel, or may be an organ. The optical part may include a reflective concave surface to have a light focusing function. The optical part may include a first surface reflecting light of wavelengths from red to near-infrared. The first surface including the reflective function can be constructed using a thin metal film, a transparent conductive film, a dielectric multilayer film, a multilayer interference film, or the like. A typical size of the optical part may be circular, elliptical, polygonal, or the like with a diameter of 10 mm to 100 μm.

Various methods, such as infrared absorption can be used as noninvasive methods for detecting components in blood, with Raman spectroscopy as one of the most suitable methods. The implant may include an optical part (optical unit) that is highly reflective for light of wavelengths from red to near-infrared so as to reflect Raman scattered light. The implant may be a transparent member, such as glass with a reflective function, or may be made of a flexible member made of resin such as silicon-based resin, a biomaterial, or the like. The implant may be a biosoluble resin (resin that dissolved in vivo), metal, or the like that dissolves after a predetermined time or period has elapsed.

The irradiating apparatus of the analyzer apparatus used in combination with the implant may include a focusing apparatus (focusing unit) that focuses at least two laser beams, for example, Stokes light and pump light for generating Raman scattered light, onto a common spot on the target either directly or via the optical part of the implant. The focusing apparatus (condensing device) may include a micromirror array to control the wave front. The analyzer apparatus may further include an optical tweezers device that forms an optical trap inside the target to be inspected. The analyzer apparatus may further include an electromagnetic field generator apparatus for performing microfluidic control of fluid inside the target.

Another aspect of the present invention is a living body monitoring apparatus kit (apparatus set, assembly kit, or biomonitoring system) including the implant and the analyzer apparatus described above. By efficiently collecting scattered light outside the skin using an implant that has been embedded in an living body, it is possible to accurately detect targets or components flowing within the target such as various components in blood in a non-invasive manner. This means that it is possible to provide a living body monitoring system capable of accurately and continuously monitoring information on the living body without selecting a method that imposes a burden (biologic load, impact, harmfulness, potentiality of harmful) on the living body, such as greatly increasing the laser intensity.

Another aspect of the present invention is a biomedical management apparatus kit including the living body monitoring apparatus kit described above and an injection apparatus that injects a drug into the living body through the skin based on the condition of the living body obtained by the analyzer apparatus. It is possible to provide a biomedical management system where, by inserting (embedding) the implant in the living body, attaching the analyzer apparatus to the surface (skin surface) of the living body, and further attaching the injection apparatus to the skin, it is possible to accurately and continuously monitor information on the living body non-invasively through various components in the blood without imposing a burden on the living body and possible, based on such monitoring, to inject a desired drug into the living body at the required time and in a required amount.

Another aspect of the present invention is a method of monitoring a condition of the living body described above. This method includes embedding an implant under the skin of the living body so as to guide light irradiated from outside the skin toward a target under the skin and/or to guide light from the target toward an outside of the skin; and detecting scattered light from the target produced by irradiating the target with a laser using an analyzer apparatus attached to the outside of the skin. The detecting includes guiding at least part of the laser light and/or at least part of the scattered light using the implant. The method may include injecting a drug into the living body through the skin using an injection apparatus based on the condition of the living body obtained by the analyzer apparatus.

### BRIEF DESCRIPTION OF DRAWINGS

FIG. 1 is a diagram depicting one example of a biomedical management system attached to the skin.

FIG. 2 is a diagram depicting another example of a biomedical management system attached to the skin.

FIG. 3 is a diagram depicting one example of controlling a wavefront.

FIG. 4 is a flowchart depicting the overall operation of a biomedical management system.

### DESCRIPTION OF EMBODIMENTS

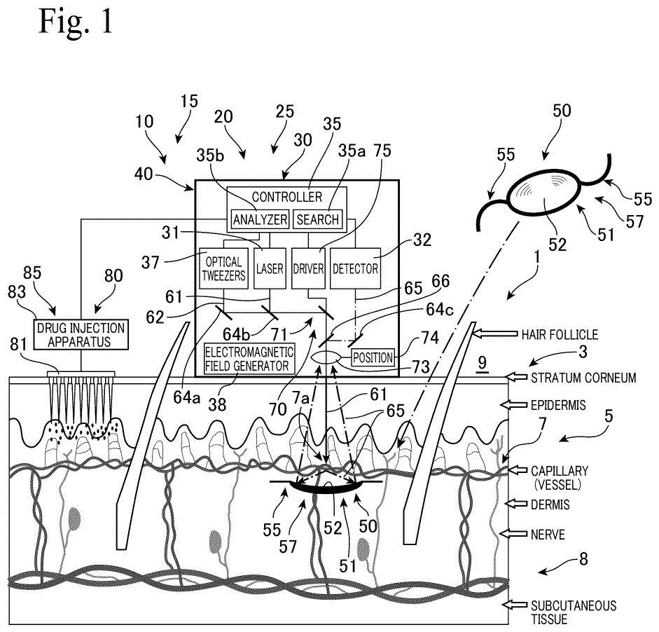

FIG. 1 depicts one example of a biomedical management system (management system of living body, health management system) 10 for managing the health condition of a living body (an organism), for example, a human body 1. This biomedical management system 10 includes a living body monitoring system (organism monitoring system, biomonitoring system) 20 that monitors the condition of the living body 1 and a medication system 80 that injects one or more drugs for maintaining the health of the living body 1. The living body monitoring system 20 is provided by a living body monitoring apparatus kit 25 including an implant 50 to be embedded (inserted) in the living body 1 and an analyzer apparatus 30 that monitors the condition of the living body 1 in combination with the implant 50. One example of the analyzer apparatus 30 is a wearable mobile terminal 40, such as a smartwatch, in which a communication function and a user interface are incorporated. The medication system 80 is provided by a medication kit (injection kit) 85 including an injector 81 that injects one or more drugs through the skin 5 of the living body 1 and a supplying apparatus (supplying unit) 83 that supplies one or more predetermined drugs to the injector 81. The biomedical management system 10 is provided by a biomedical management apparatus kit 15 including the living body monitoring apparatus kit 25 and a medication kit (injection kit) 85.

One example of the implant 50 is an implant that is embedded under the skin 5 of the living body 1, and includes an optical part (optical unit) 51 that has a reflective function and is implanted so as to be positioned below a target under the skin, typically a blood vessel 7 containing the blood that is the target. Although the measurement target is the blood itself carried in a blood vessel, the following description includes focusing light on a blood vessel 7 as the irradiation target for measurement.

The implant 50 includes the optical part 51 that guides light 61 emitted from outside 9 of the skin (that is, outside the epidermis 3) to the target (as one example, a blood vessel) 7 under the skin and/or guides light 65 from the target 7 to the outside 9 of the skin. With this implant 50, it is also possible to control the direction of propagation of light under the skin (that is, inside the living body) 8 so as to focus light toward a target (for example, a blood vessel) 7 inside the body (in vivo) 8 and to guide light from the target 7 to the outside of the skin 9. The target may be tissue under the skin, such as a capillary 7 under the epidermis 3, may be tissue, such as a blood vessel, present in the dermis or subdermal subcutaneous tissue, or may be a blood vessel or tissue at a deeper location.

The implant 50 may further include a support portion 55 for maintaining the position of the optical part 51 inside the body 8. One example of the optical part 51 may be a unit with a circular, elliptical, or polygonal concave surface 52 with a diameter of 100 μm to 10 mm. The concave surface 52 has a reflective function, and focuses or collimates light by reflecting incident light 61 or the scattered light 65 at the concave surface 52.

One example of the support portion 55 that supports the optical part 51 is one or more parts that partially protrudes from the perimeter of the optical part 51 so that the concave surface 52 is maintained inside the body 8 in a state facing the blood vessel 7 that is the target. The support portion 55 may be one or a plurality of arm-shaped members that extend in an S-shaped or J-shaped curve. The concave surface 52 of the optical part 51 includes a reflective surface (first surface), and may be a surface provided with reflective performance using at least one of thin metal films, transparent conductive films, dielectric multilayer films, and a multilayer interference film. The transparent conductive film, the dielectric multilayer film, and the multilayer interference film may be designed to have a high reflective performance (that is, reflectance) for light with wavelengths from red to near-infrared.

The optical part 51 itself may be made of a material, such as metal, with a reflective function. The implant 50 may be made of silicone resin or other resins that have a high affinity with the living body 1 and do not or hardly obstruct tests such as an MRI, a metal such as titanium, or a suitable biomaterial. The implant may be a biosoluble (soluble in vivo) resin or metal that dissolves in the living body 8 after a predetermined time or period required for observing the living body 1 has elapsed.

The implant 50 may be embedded in the living body 1 by a simple operation, or an implant 50 made of a highly flexible material may be inserted from the surface of the living body into a suitable position inside the living body using a suitable injection device, or a needle for drip infusion or catheter. The implant 50 may be embedded in a position where it is possible to irradiate the blood vessel (blood capillary) 7 below the skin with the light 61 and control the scattered light 65 from the blood vessel 7, but there are no particular limitations on the location where the implant 50 is embedded in the organism or living body 1. If the analyzer apparatus 30 is implemented in a mobile terminal 40 such as a smartwatch, the implant 50 may be embedded at a location where the mobile terminal 40 is attached, as one example, under the skin where a smartwatch is worn on the wrist.

The implant 50 may further include a marker 57 for enabling contactless detection of the position within the living body of the optical part 51 from outside the living body. One example of the marker 57 is ceramic or resin including a metal or magnetic material that can be detected from outside the skin using a magnetic field. The marker 57 may be included in part or all of the optical part 51. Part or all of the support portion 55 may be the marker 57, or a part including the marker 57 may be independently formed around the optical part 51.

One example of the analyzer apparatus 30 is a non-invasive analyzer apparatus that uses one or more lasers, and acts in cooperation with the optical part 51 of the implant 50 to accurately acquire various information related to or included in the target, in the present embodiment, blood flowing through the blood vessel 7. Although various methods, such as infrared absorption, can be used for the analyzer apparatus 30, the analyzer apparatus 30 in the present embodiment uses a laser light source (laser apparatus or laser unit) 31 that generates the lasers 61 that are incident on the blood vessel 7 either directly or in cooperation with the implant 50, and a detector (detection unit) 32 for detecting the scattered light 65 from the blood vessel 7 either directly or in cooperation with the implant 50. The laser apparatus 31 emits at least two types of laser beam, in this example, the laser lights 61 including Stokes light and pump light for generating Raman scattered light (CARS (Coherent Anti-Stokes Raman Scattering) light). The laser apparatus 31 may be an apparatus that emits probe light in addition to Stokes light and pump light as the laser light 61.

The analyzer apparatus 30 further includes an irradiating apparatus 70 which irradiates the various types of laser light 61 onto a common spot on the blood vessel 7 either directly or in cooperation with the implant 50. The irradiating apparatus 70 has a function as a focusing apparatus (focusing unit) for collimating the emitted laser light 61 and collecting the scattered light 65 from the target. The focusing apparatus 70 includes an objective lens 73, a micromirror array (micromirror device) 71 that controls the wave front of the laser lights 61, and a driver 75 that controls the micromirror array 71. The focusing apparatus 70 may be equipped with a control apparatus (irradiation position control apparatus or irradiation position control optical system) 74 that performs control to guide the laser lights 61 to the position of the implant 50 embedded under the skin (that is, inside the body) 8 in order to detect (measure) the composition of the blood inside the blood vessel. The irradiation position control apparatus 74 may have a function of controlling the irradiation direction of the laser lights 61 so as to guide the scattered light 65 produced by irradiation with the laser lights 61 via the implant 50 to outside 9 of the skin. The irradiation position control apparatus 74 may be an apparatus that controls the position and orientation of the objective lens 73, or may be an apparatus that controls the irradiation direction and angle of the laser using a reflective apparatus such as a digital mirror device.

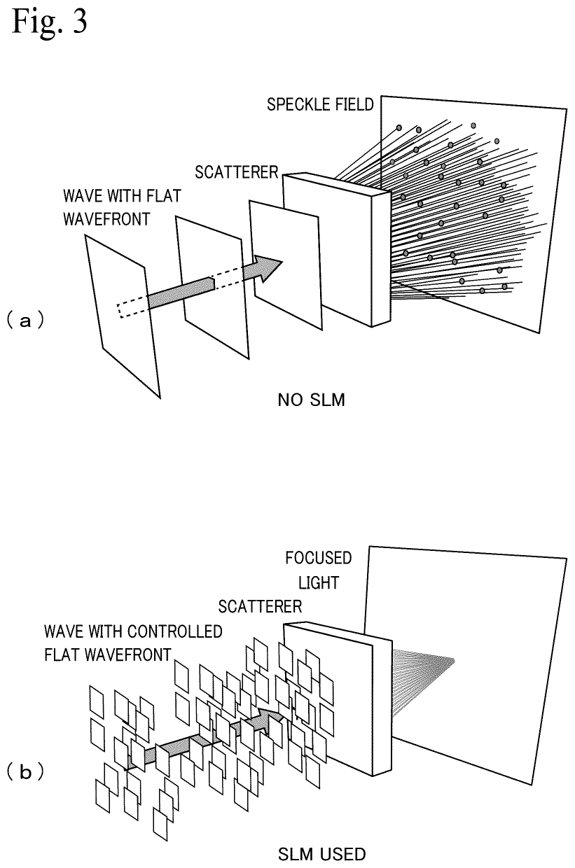

As depicted in FIG. 3(a), the skin 3 is a scatterer (diffusive material), and laser light is scattered by random media when a light beam with a flat wave front passes through it. For this reason, a pattern of bright and dark spots called speckles may be observed in the scattered light. However, it is possible to use a spatial light modulator (SLM), such as the micromirror array device 71, to control the wave front so as to cancel such scattering caused by a scatterer. As a result, as depicted in FIG. 3(b), by the objective lens 73 or by the cooperation of the objective lens 73 and the concave optical part 51 of the implant 50, the laser lights 61 can be focused effectively onto a predetermined spot on the blood vessel 7, which is the target, in a state where various aberrations are suppressed. The micromirror array device 71 may be controlled via the driver 75 by a controller 35 so as to maximize the intensity of scattered light to be detected for example. Alternatively, the controller 35 may move the micromirror array device 71 in a predetermined pattern or a random pattern via the driver 75 to search for a pattern that maximizes the intensity of the obtained scattered light.

The controller 35 of the analyzer apparatus 30 may be further provided with a search apparatus (search function) 35a that searches for the specific (detailed) position of the implant 50 that has been inserted inside the living body 8 under the skin and controls where the laser beams 61 are irradiated. One example of the search apparatus 35a may have a function that uses an electromagnetic field generator apparatus 38 to detect the marker 57 of the implant 50 using an electromagnetic field. The search apparatus 35a may include an image processing function, such as OCT, that detects the position of the implant 50 inside the living body 8. The search apparatus 35a may also be an apparatus that scans an intended implanting position of the implant 50 and the periphery with the laser light 61 and determines the position of the implant 50 based on whether the scattered light 65 includes information of blood components, such as glucose. The search apparatus 35a may implement processing (preprocessing) that determines the position of the implant 50 and decides the irradiation position of the laser lights 61 before the start of the measurement, intermittently during measurement, or in parallel with the measurement.

One example of a spectroscopy-type analysis module (analyzer apparatus) 30 is a Raman analyzer apparatus. As specific examples, a CARS (Coherent Anti-Stokes Raman Scattering) analyzer apparatus, an SRS (Stimulated Raman Scattering) analyzer apparatus, and a time-resolved CARS analyzer apparatus that are suited to microanalysis may be used.

The analyzer apparatus 30 includes a controller (control apparatus or control unit) 35 equipped with a function 35b that controls the irradiating apparatus 70 and analyzes a measurement result produced by the scattered light 65 obtained by the detector 32, and a function 35a that controls the irradiating apparatus 70 and the detector 32 as a search apparatus. The controller 35 may further include a function (communication function) for providing measurement results to the outside, for example an external system such as a cloud-based health care server, a function for cooperating with the medication system 80, and the like. The controller 35 may be equipped with computer resources, such as a memory and a CPU, and control the analyzer apparatus 30 and the living body monitoring system 20 and/or biomedical management system 10 including the analyzer apparatus 30 using a program (program product).

The analyzer apparatus 30 may further include an optical tweezers apparatus 37 that forms an optical trap inside the target blood vessel 7. Optical tweezers use an objective lens with a high numerical aperture to focus laser light to the greatest extent possible, and generates a force that traps micrometer-sized particles using momentum transfer caused by photon scattering. Particles or molecules of a predetermined size can be trapped from the blood flowing through the blood vessel 7 and subjected to Raman spectroscopic analysis.

The analyzer apparatus 30 may further include an electromagnetic field generator apparatus 38 for microfluidic control of the fluid within the blood vessel or capillary 7. A structure that traps and/or filters molecules or particles in the blood, such as a nano-sized molecular sieve, a NanoPen chamber, or the like can be dynamically formed in the blood vessel 7 by microfluidic control of the electromagnetic field generator apparatus 38 and/or optical tweezers (an optical trap), or cooperation between such elements through control by the controller apparatus 35.

Examples of molecules that can be captured or found by the analyzer apparatus 30 via the implant 50 are not limited to blood cells, such as red blood cells, white blood cells, lymphocytes, and platelets, and include all molecules that may be present in the blood, such as antibodies, antibody fragments, genetically modified antibodies, single-chain antibodies, receptor proteins, binding proteins, enzymes, inhibitor proteins, lectins, cell adhesion proteins, oligonucleotides, polynucleotides, nucleic acids, and aptamers. Example targets of detection and/or identification by the monitoring system 20 including the implant 50 and the analyzer apparatus 30 include any atom, chemical substance, molecule, compound, composition, microorganism or aggregate, such as blood cells, amino acids, peptides, polypeptides, proteins, glycoproteins, lipoproteins, nucleosides, nucleotides, oligonucleotides, nucleic acids, sugars, carbohydrates, oligosaccharides, polysaccharides, fatty acids, lipids, hormones, metabolites, cytokines, chemokines, receptors, neurotransmitters, antigens, allergens, antibodies, substrates, metabolites, cofactors, inhibitors, drugs, pharmaceuticals, nutrients, prions, toxins, poisons, explosives, pesticides, chemical warfare agents, biological hazards, radioisotopes, vitamins, heteroaromatics, carcinogens, mutagens, narcotics, amphetamines, barbiturates, hallucinogens, waste, and/or contaminants, but are not limited to such. Microorganisms include, but are not limited to, viruses, bacteria, cells, and the like.

In the living body monitoring system 20 depicted in FIG. 1, laser lights 61 for Raman spectroscopy (Stokes light, pump light and probe light) 61 outputted from the laser unit 31 of the analyzer apparatus 30 attached to the outside 9 of the skin and laser light 62 of the optical tweezers apparatus 37 are collected by optical elements 64a and 64b, such as mirrors, and irradiated onto the target blood vessel 7 under the skin 8 by the irradiating apparatus (emitting apparatus, focusing apparatus) 70. In the irradiating apparatus 70, the laser lights 61 are focused via the objective lens 73 onto a predetermined position (spot) 7a on the blood vessel 7 under the skin after the wave front has been controlled by the micromirror device 71. Scattered light (CARS light) 65 including information on the blood flowing through the blood vessel 7 is outputted in various directions from the spot 7a. The CARS light 65 emitted forward with the highest intensity (front CARS light), that is, forward in the direction of incidence of the laser lights 61, is reflected by the concave surface (reflecting surface) 52 of the optical part 51 of the implant 50, which is embedded in advance below the spot 7a of the blood vessel 7, in a direction toward the outside 9 of the skin and is collected through the objective lens 73 of the analyzer apparatus 30. The CARS light 65 is guided to a detector (spectrometer) 32 via suitable optical elements 66 and 64c. Since CARS light outputted backward (epi-CARS light) in the incident direction of the laser light 61 of the spot 7a is outputted toward the skin, such light is also collected through the objective lens 73 of the analyzer apparatus 30 and guided to the detector 32.

For this reason, in this living body monitoring system 20, the CARS light 65 scattered inside the body can be collected in the direction of the analyzer apparatus 30 by the implant 50, so that the CARS light 65 can be detected more efficiently. Accordingly, by attaching the analyzer apparatus 30 to the surface of the living body 1 so that the lasers 61 irradiate the spot 7a on the blood vessel 7 above the implant 50, it is possible to provide the living body monitoring system 20 that detects various components in the blood non-invasively with high accuracy. Also, by using the living body monitoring apparatus kit 25, it is possible to provide the living body monitoring system 20 capable of accurately and continuously monitoring information on the living body without imposing a burden on the living body.

The medication system (dosing system, dispensing system) 80 includes a drug injecting apparatus (supplying apparatus) 83, which supplies (injects) an amount of one or more drugs required to maintain the health of the living body 1 in predetermined states at the required timing based on the measurement results of the analyzer apparatus 30, and an injector 81 which is attached to the skin 5 to inject the drug or drugs. The injector 81 may be an injector that uses microneedles, or may be a needleless injector that injects a drug solution through the skin by spraying without using a needle.

It is possible to provide a biomedical management apparatus kit (that is, a set for assembling a biomedical management apparatus) 15 including the living body monitoring apparatus kit (living body monitoring kit) 25 and a medication kit 85 for injecting a drug into the living body 1 through the skin 5 based on the condition of the living body 1 obtained by the analyzer apparatus 30. According to this biomedical management apparatus kit 15, the implant 50 is embedded in the living body 1, the analyzer apparatus 30 is attached to the surface (the skin surface or skin) 9 of the living body 1 so that the CARS light 65 produced by the lasers 61 can be efficiently acquired via the implant 50, and the injector 81 of the medication system 80 is attached to the skin 5 and in particular to the epidermis 3, thereby attaching the biomedical management system 10 to the living body 1. With the biomedical management system 10, it is possible to accurately and continuously monitor information on the living body 1 via various components in the blood in a non-invasive manner without imposing a burden on the living body 1, and based on this, to inject a desired drug and/or drugs into the living body only in the required amount and at the required time.

In a biomedical management system (health management system) 10, in one example, the analysis system (monitor) 20 can continuously and accurately measure blood glucose in real time. Accordingly, doses of insulin can be finely controlled by the medication system 80 based on the glucose concentration that is continuously measured. In addition, the biomedical management system 10 may be equipped with a function such as an event recognition module that can predict or grasp the behavior or lifestyle of a living body (human body). The biomedical management system 10 detects various behavior including the occurrence of events (patient activities), such as exercise and meals, and predicts patient activities using a daily schedule and the outputs of various sensors. The type and amount of drug or drugs to be administered, for example insulin, may be determined so as to correspond to a predicted condition. As one example, the blood glucose concentration can be controlled within a narrow range where there is little effect on health. Accordingly, by attaching the biomedical management system 10, it is possible for a diabetic patient to play sports and have meals in the same way as a nonaffected person.

The physiologically active substance injected from the injection system (medication system or drug delivery system) 80 is not limited to insulin, and may be other hormones, prescription drugs, minerals, nutrients, or the like and the biomedical management system 10 may include a function (dosage estimation function) for determining and controlling the types and amounts of such substances. In addition, the biomedical management system 10 may be equipped with a system for sharing real-time information on the living body acquired by the system 10 and medication information at any time or continuously with an external monitoring system, such as a medical or insurance system.

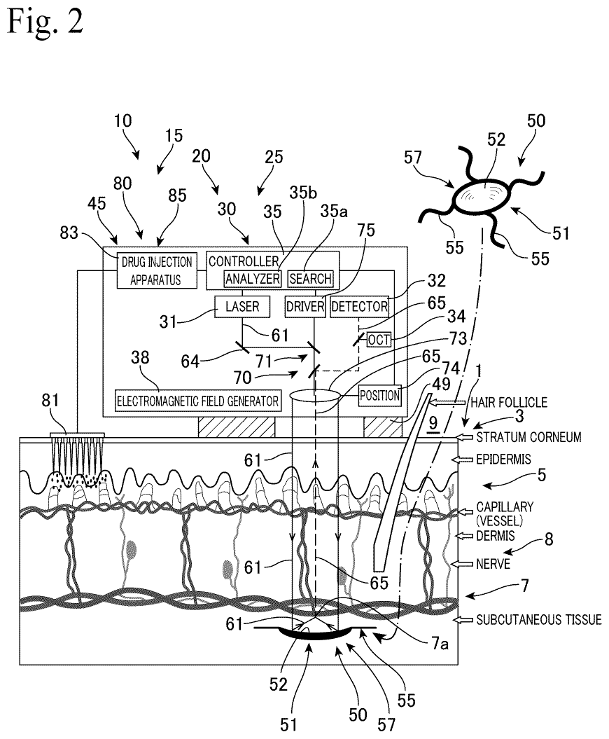

FIG. 2 depicts a different example of a living body monitoring system 20. One example of the analyzer apparatus 30 is a dedicated terminal (specialized device, application specific device) 45 for providing the biomedical management system 10. This dedicated terminal 45 may incorporate a function as the medication system 80 in addition to a function as the analyzer apparatus 30, and may be capable of attachment to the skin 5 using an adhesive pad 49 or the like. There are no limitations on the method of attaching the terminal 45, and any method that enables the terminal 45 to be fixed at a predetermined position on the skin 5 may be used.

In this example, laser lights for Raman spectroscopy (Stokes light, pump light and probe light) 61 outputted from the laser unit 31 of the analyzer apparatus 30 attached to the skin 5 are guided via an optical element 64, such as a mirror, to the irradiating apparatus 70, the wave front of the lights controlled by the micromirror device 71 of the irradiating apparatus (focusing apparatus) 70, and the light is then emitted or irradiated via the objective lens 73 into the living body 8 under the skin. Inside the body 8, the laser lights 61 are reflected by the concave surface 52 of the optical part 51 of the implant 50, which has been inserted below the blood vessel 7 in the skin (for example, the dermis) 5, and are focused in vivo onto the spot 7a on the blood vessel 7.

Accordingly, by attaching the analyzer apparatus 30 to the surface of the living body 1 so that the lasers 61 are irradiated onto the implant 50, various components in the blood can be detected non-invasively and with high accuracy. This makes it possible to provide the living body monitoring system 20 capable of accurately and continuously monitoring information on the living body without imposing a burden on the living body 1. That is, since the lasers 61 are focused by the implant 50 in vivo (inside the body), the spot of the lasers 61 reaching the implant 50 through the skin 3 or 5 can be enlarged (not focused). This means that the intensity (illuminance or intensity per unit area) of the lasers 61 passing inside the body 8 can be reduced, which makes it possible to reduce the burden (biologic load, impact, potentially of harmful) on the living body 1, including the skin 3. On the other hand, at the implant 50 inside the body 8, the laser 61 can be focused toward a predetermined spot 7a from a location near the blood vessel 7 that is the target. Accordingly, the blood vessel 7 that is the target can be irradiated with a sufficiently high-intensity lasers 61 while minimizing the effect on the living body 1, and as a result, CARS light 65 of a sufficiently high-intensity can be generated and detected.

Scattered light (CARS light) 65 including information about the blood flowing through the blood vessel 7 is outputted in various directions from the spot 7a. In the present embodiment, the implant 50 located below the blood vessel 7 irradiates the blood vessel 7 with laser beams 61 from the bottom (lower side) toward the outside 9 of the skin (upper side). This means that the CARS light (front CARS light) 65 outputted to the front where the CARS light 65 has the highest intensity, that is, forward in the incident direction of the laser lights 61, is outputted in the direction of the skin (the direction above 9 the skin 5). Accordingly, this high-intensity CARS light 65 can be acquired or collected through the objective lens 73 of the analyzer apparatus 30 and can be efficiently analyzed by the detector (spectrometer) 32. Since the CARS light (epi-CARS light) outputted backward in the incident direction of the laser lights 61 at the spot 7a is reflected toward the skin by the concave surface 52 of the optical part 51 of the implant 50, such light can also be acquired through the objective lens 73 of the analyzer apparatus 30. This means that light including the CARS light 65 that was scattered to the rear may be detected by the detector 32 as well.

The analyzer apparatus 30 may include, as an apparatus for detecting the position of the implant 50 embedded under the skin 8 of the living body 1, an OCT 34 which is capable of optical imaging of substances and locations in the body. If the implant 50 is formed of a shape or material that can be identified as a substance that differs from other tissue in the living body 8, the search function 35a can use the OCT 34 to confirm the position of the implant 50 in advance or during every usage and irradiate the lasers 61 with high precision so that the CARS light 65 can be efficiently obtained.

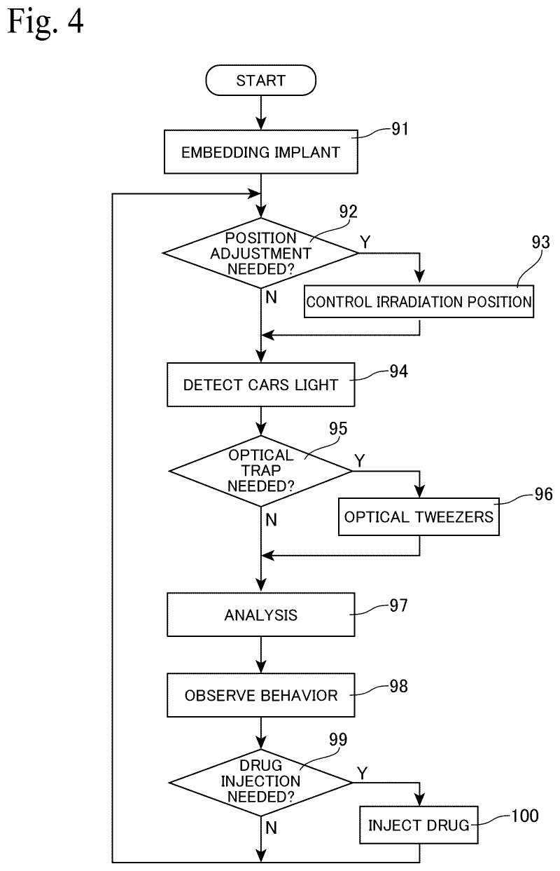

FIG. 4 depicts an overview of a process (method) for monitoring the condition of the organism or living body 1 using the system 10 or 20 described above. First, in step 91, the implant 50 is embedded under the skin (that is, inside the body, in vivo) 8 of the living body 1. In more detail, the implant 50 is inserted under the skin 8 of the living body 1 so that the lights 61 irradiated from outside 9 of the skin are directed at a target under the skin (the blood vessel 7, or more specifically, the blood within the blood vessel) and/or light (scattered light or CARS light) 65 from the target is guided toward the outside 9 of the skin. The implant 50 may be embedded in advance by a simple surgical operation, or may be installed by the patient himself/herself using a jig for inserting the implant 50. The implant 50 may be made of a material that dissolves or is absorbed by the living body 1, and may be periodically implanted.

In step 94, by the analyzer apparatus 30 that has been attached to the surface of the living body, for example, outside 9 of the skin, the inside of the body 8 is irradiated with the laser 61 and the scattered light (CARS light) 65 obtained from the target (which is the blood in the blood vessel) is detected (obtained). At least some of the laser lights 61 and/or at least some of the CARS light (scattered light) 65 is guided by the implant 50, which results in the CARS light 65 being efficiently detected by the analyzer apparatus 30.

As one example, in the example depicted in FIG. 1, the monitoring process includes implanting or embedding the implant 50 under the skin 8 of the living body 1 so as to be below the blood vessel 7, and using the analyzer apparatus 30 attached to the surface 9 of the living body to irradiate the blood vessel 7 with the lasers 61 and detect the scattered light (CARS light) 65 obtained from the blood vessel 7 via (reflected by) the implant 50. In the example depicted in FIG. 2, the monitoring process involves embedding the implant 50 under the skin (inside the body) 8 of the living body 1 so as to be below the blood vessel 7 and the analyzer apparatus 30 attached to the living body surface 9 irradiating the blood vessel 7 with the laser 61 via the implant 50 and detecting the scattered light (CARS light) 65 obtained from the blood vessel 7.

Prior to the processing in step 94, in step 92, a search for the position of the implant 50 may be performed to determine, by pre-analyzing the scattered light 65 for example, whether adjustment of the irradiation position is necessary. If adjustment of the irradiation position is necessary, in step 93, the detailed position of the implant 50 is searched using the search apparatus 35a.

If, in step 94, CARS light 65 is detected and it is necessary in step 95 to form an optical trap in the blood vessel 7 that is the target, in step 96 an optical trap is formed by the optical tweezers apparatus 37. In step 97, the CARS light 65 detected via the implant 50 is analyzed to obtain information relating to the targeted components in the blood. In addition, in step 98, the biomedical management system 10 may perform process using other functions of the mounted wearable terminal, for example, monitoring or observing the behavior of the living body (user) 1 based on information from an accelerometer or the like, and/or information from another wearable terminal and also information obtained from an Internet (cloud) server or the like.

In step 99, it is possible to determine whether medication is needed based on the information obtained by the analyzer apparatus 30 and also the information obtained by observing behavior, and in step 100, injection of a drug or drugs using the medication system 80 may be performed. The medication system 80 may inject a drug or drugs into the living body 1 through the skin using an injection apparatus (injector) 81 based on the condition of the living body 1 obtained by the analyzer apparatus 30.

The method of monitoring the condition of a living body, including the processes described above, may be stored on a computer-readable recording medium and provided as a program (program product) for controlling the monitoring system 20 including the analyzer apparatus 30. Also, the method of monitoring the condition of a living body may be provided as a program that is downloadable via the Internet or the like, or may be provided as a service via the Internet.

The above discloses an implant to be embedded under the skin of an organism (living body), the implant having an optical part or unit with a reflective function that is inserted below a target under the skin. The implant may further include a support portion for maintaining the position of the optical unit inside the body. The implant may further include a marker portion for enabling contactless detection of the position of the optical unit inside the body from outside the body. The target may include a blood vessel. The optical unit may include a concave surface with a reflecting function, and the optical unit may include a first surface with a reflecting function for light of wavelengths from red to near-infrared. The first surface may include a surface on which at least one of a thin metal film, a transparent conductive film, a dielectric multilayer film, and a multilayer interference film is formed. The optical unit may be circular with a diameter of 10 mm to 100 μm.

The above description also discloses an analyzer apparatus including an irradiating apparatus that irradiates the target under the skin of the organism with one more lasers, and a detector that detects scattered light from the target via the optical unit of the implanted implant. The irradiating apparatus may include a focusing apparatus for focusing at least two laser beams onto a common spot on the target. The above description also discloses an analyzer apparatus including an irradiating apparatus that irradiates the target with a laser via the optical unit of an implant that has been implanted under the skin of an organism, and a detector that detects scattered light from the target. The irradiating apparatus may include a focusing apparatus for focusing at least two laser beams via the optical unit of the implant onto a common spot on the target. The focusing apparatus may include a micromirror array that controls the wave front. The analyzer apparatus may further include an apparatus for detecting the position of the implant implanted under the skin of the organism.

The above description also discloses an organism monitoring apparatus kit including the implant described above and the analyzer apparatus described above. The above description also discloses a biomedical management apparatus kit including an injection apparatus for injecting a drug into the organism through the skin based on a condition of the organism obtained by the analyzer apparatus.

The above description also discloses a method of monitoring the condition of an organism. This method includes embedding an implant with a reflective function below a target under the skin of the organism, and an analyzer apparatus irradiating the target with a laser and detecting scattered light obtained from the target including reflected light from the implant. In addition, this method may include embedding an implant with a reflective function below the target under the skin of the organism, and an analyzer apparatus irradiating the target with a laser via the implant and detecting scattered light obtained from the target. The method may also include injecting a drug into the organism via the skin with an injection apparatus based on the condition of the organism obtained by the analyzer apparatus.

Note that although specific embodiments of the present invention have been described above, various other embodiments and modifications will be conceivable to those of skill in the art without departing from the scope and spirit of the invention. Such other embodiments and modifications are addressed by the scope of the patent claims given below, and the present invention is defined by the scope of these patent claims.

1. An implant to be embedded under a skin of a living body, the implant comprising:
an optical part that focuses light irradiated from outside the skin on a target under the skin and/or guides light from the target toward the outside of the skin to focus on the outside.

2. The implant according to claim 1,
wherein the optical part includes a reflective surface and is embedded so as to be positioned below the target.

3. The implant according to claim 1,
further comprising at least one support portion that partially protrudes from a perimeter of the optical part and maintains a position inside the living body of the optical part.

4. The implant according to claim 1,
further comprising a marker that enables contactless detection of a position inside the living body of the optical part from outside the living body.

5. The implant according to claim 1,
wherein the optical part includes a reflective concave surface.

6. The implant according to claim 1,
wherein the optical part includes a first surface reflecting the light of wavelengths from red to near infrared.

7. The implant according to claim 6,
wherein the first surface includes a surface formed of at least one of a thin metal film, a transparent conductive film, a dielectric multilayer film, and a multilayer interference film.

8. The implant according to claim 1,
wherein the optical part has a circular, elliptical, or polygonal shape with a diameter of 100 μm to 10 mm.

9. The implant according to claim 1,
wherein the target is blood flowing through a blood vessel, and
the implant is embedded so that the optical part focuses light irradiated from outside the skin on the blood vessel and/or guides light from the blood inside the blood vessel the outside of the skin.

10. An analyzer apparatus that is attached to a skin of a living body, the analyzer apparatus comprising:
an irradiating apparatus that irradiates a target under the skin of the living body with one or more lasers; and
a detector that detects scattered light from the target via the optical part of the implant according to claim 1 that has been embedded under the skin of the living body.

11. The analyzer apparatus according to claim 10,
wherein the irradiating apparatus includes a focusing apparatus that focuses at least two laser beams on a common spot on the target.

12. An analyzer apparatus that is attached to the skin of a living body, the analyzer apparatus comprising:
an irradiating apparatus that irradiates a target via the optical part of the implant according to claim 1 that has been embedded under the skin of the living body; and
a detector that detects scattered light from the target.

13. The analyzer apparatus according to claim 12,
wherein the irradiating apparatus includes a focusing apparatus that focuses at least two laser beams on a common spot on the target via the optical part.

14. The analyzer apparatus according to claim 11,
wherein the focusing apparatus includes a micromirror array that controls a wave front.

15. The analyzer apparatus according to claim 10,
further comprising an apparatus that detects a position of the implant that has been embedded under the skin of the living body.

16. A living body monitoring system comprising the analyzer apparatus according to claim 10.

17. A biomedical management system comprising:
the analyzer apparatus according to claim 10; and
an injection apparatus that injects a drug into the living body via the skin, based on a condition of the living body obtained by the analyzer apparatus.

18. A living body monitoring apparatus kit comprising:
an implant to be embedded under a skin of a living body, the implant comprising an optical part that guides light irradiated from outside the skin toward a target under the skin or guides light from the target the outside of the skin; and
the analyzer apparatus according to claim 10.

19. A biomedical management apparatus kit comprising:
the organism monitoring apparatus kit according to claim 18; and
an injection apparatus that injects a drug into the living body via the skin based on a condition of the organism obtained by the analyzer apparatus.

20. A method of monitoring a condition of a living body comprising:
embedding the implant according to claim 1 under the skin of the living body so as to focus light irradiated from outside the skin on a target under the skin and/or to guide light from the target toward an outside of the skin to focus on the outside; and
detecting scattered light from the target produced by irradiating the target with a laser using an analyzer apparatus attached to the outside of the skin,
wherein the detecting includes guiding at least part of the laser light and/or at least part of the scattered light using the implant.

21. The method according to claim 20,
further comprising an injection apparatus attached to a surface of the living body injecting a drug into the living body via the skin based on a condition of the living body obtained by the analyzer apparatus.
